(19) United States
(12) Patent Application Publication
(10) Pub. No.: US 2008/0181943 A1
(43) Pub. Date: Jul. 31, 2008

(54) COMBINATION OF A LONG-ACTING HYPNOTIC AGENT AND A SHORT-ACTING HYPNOTIC AGENT AND THERAPEUTIC USE THEREOF

(75) Inventors: Alain CUINE, Saint Fargeau-Ponthierry (FR); Michel DECOBERT, Gif Sur Yvette (FR); Dominique FRANCON, Plaisir (FR); Henry SAUNAL, Montpellier (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/029,011

(22) Filed: Feb. 11, 2008

Related U.S. Application Data
(63) Continuation of application No. PCT/FR2006/001830, filed on Jul. 27, 2006.

(30) Foreign Application Priority Data
Aug. 19, 2005 (FR) 0508643

(51) Int. Cl. A61K 9/48; A61K 31/437; A61K 9/28

(52) U.S. Cl. 424/456; 514/300; 424/468; 424/472

(57) ABSTRACT

The invention relates to the combination of: a short-acting hypnotic agent which is selected from among a modulators of receptors GABA-A, a benzodiazepine, a phenothiazine, a melatonin derivative and a melatonin receptor agonist; and a long-acting hypnotic agent which is selected from among a modulator of receptors GABA-A, a benzodiazepine, an antagonist of receptors 5HT2A and a calcium ion modulator, for the treatment of sleep disorders. The invention also relates to galenic formulations containing said combinations.

This application is a continuation of International application No. PCT/FR2006/001,830, filed Jul. 27, 2006, which is incorporated herein by reference in its entirety; which claims the benefit of priority of French Patent Application No. 05/08,643, filed Aug. 19, 2005.

The invention relates to a combination of at least one long-acting hypnotic agent and of at least one short-acting hypnotic agent. The invention also relates to a composition containing it and to its therapeutic use.

A certain number of hypnotic agents with varying modes and durations of action have been developed over the years.

A first category of hypnotic agents consists of those with a short duration of action. In the text hereinbelow, the term “short-acting hypnotic agent” means a compound that acts mainly as a sleep inducer, i.e. a compound that acts on the time for entering into the sleep phase.

Thus, zolpidem is a short-acting hypnotic agent, which acts as a GABA-A receptor modulator. Zolpidem belongs to the class of imidazopyridines, and is administered orally in the form of an immediate-release tablet or in a galenical form allowing delayed release.

Zolpidem acts quickly, is absorbed well and has a bioavailability of 70%. The mean dose, between 5 and 10 mg in a conventional formulation, induces a maximum plasmatic concentration that is reached between 0.5 and 3 hours, the half-life is short, with a mean value of 2.4 hours and a duration of action ranging up to 6 hours.

Other examples of short-acting hypnotic agents are zaleplon, which belongs to the class of pyrazolopyrimidines, zopiclone and eszopiclone, which belong to the class of cyclopyrrolones, and also derivatives thereof.

Long-acting hypnotic agents have also been developed. In the text hereinbelow, the term “long-acting hypnotic agent” means a compound that acts mainly on the quality and/or maintenance of sleep, especially the phases of deep sleep.

Such a long-acting hypnotic agent is, for example, eplivanserin. Eplivanserin is a 5HT2A receptor inhibitor that acts without blocking dopamine. Eplivanserin, and the preparation thereof, is especially described in document EP-A-0 373 998.

Eplivanserin is also absorbed well, with a bioavailability of 80%. The conventional dosage, between 1 and 10 mg, induces a maximum plasmatic concentration that is reached between 2 and 6 hours, the half-life time being relatively long, with a mean value of 50 hours.

Other long-acting hypnotic agents are, for example, gaboxadol and pregabaline, and also derivatives thereof.

The hypnotic agents described above allow sleep disorders to be treated, especially insomnia. However, whereas short-acting hypnotic agents act mainly on the entry into the sleep phase, long-acting hypnotic agents act rather on the phase of deep sleep.

In addition, the hypnotic agents may, especially when they are administered at high doses, have a negative impact on the awake periods, in particular that following the taking of the medicament.

It is thus still desirable to have available a composition that can induce or maintain repairing sleep, and that can do so at a low dose.

The aim of the invention is to overcome this drawback, by proposing a combination that makes it possible to combine the actions of short-acting and long-acting hypnotic agents, further improving the quality of sleep and the respective effects of the short-acting and long-acting hypnotic agents, without a negative effect on the patient's awake phases.

A first subject of the invention thus concerns a combination of two hypnotic agents.

Another subject of the invention concerns a pharmaceutical composition containing a combination of two hypnotic agents.

Another subject of the invention concerns the use of this combination for the preparation of a medicament.

According to a first aspect, the invention relates to a combination of two hypnotic agents.

The combination of the invention comprises at least one short-acting hypnotic agent and at least one long-acting hypnotic agent.

According to one embodiment, the short-acting hypnotic agent is present in a galenical form suitable for immediate or sustained release, and the long-acting hypnotic agent is present in a galenical form suitable for immediate release.

It has been discovered, specifically, that the combination of a short-acting hypnotic agent with a long-acting hypnotic agent makes it possible to obtain a beneficial effect on the patient's sleep, and that this effect is greater than that of each of the two hypnotic agents considered individually.

According to a first aspect of the invention, the short-acting hypnotic agent and the long-acting hypnotic agent are released immediately. The two agents then appear in the plasma according to their respective pharmacokinetic characteristics. Thus, the short-acting hypnotic agent appears in the plasma before the long-acting hypnotic agent.

According to this embodiment, each agent develops its mechanism of action, with a synergistic effect between the two agents.

According to a second aspect of the invention, the short-acting hypnotic agent is released in a sustained manner and the long-acting hypnotic agent is released immediately. According to this embodiment, the action time of the short-acting hypnotic agent is increased, with a longer residence time in the plasma. Thus, the two agents may act at the same time, again with a synergistic effect.

Examples of short-acting hypnotic agents that may be used in the context of the invention are especially GABA-A receptor modulators, benzodiazepines, phenothiazines, melatonin derivatives and melatonin receptor agonists.

For example, the short-acting hypnotic agent may be chosen especially from zolpidem, zopiclone, eszopiclone, zaleplon, melatonin, ramelteon, triazolam, etizolam, brotizolam and indiplon, and also derivatives and/or mixtures thereof.

Examples of long-acting hypnotic agents that may be used in the context of the invention are especially 5HT2A receptor antagonists, GABA-A receptor modulators, benzodiazepines and calcium-ion modulators.

For example, the long-acting hypnotic agent may be chosen especially from eplivanserin, temazepam, clonazepam, gaboxadol and pregabaline, and also derivatives and/or mixtures thereof.

The short-acting or long-acting hypnotic agents described above may comprise one or more asymmetric carbon atoms. They may thus exist in the form of enantiomers or diastereoisomers. These enantiomers and diastereoisomers, and also mixtures thereof, including racemic mixtures, form part of the invention.

The short-acting or long-acting hypnotic agents described above may also exist in the form of bases or of acid-addition salts. Such addition salts form part of the invention.

These salts may be prepared with pharmaceutically acceptable acids.

The short-acting or long-acting hypnotic agents described above may also exist in the form of hydrates or solvates, i.e. in the form of associations or combinations with one or more water molecules or with a solvent. Such hydrates and solvates also form part of the invention.

According to one implementation form of the invention, the combination comprises zolpidem, especially in hemitartrate form, as short-acting hypnotic agent, and eplivanserin, especially in fumarate form, as long-acting hypnotic agent.

According to another aspect, the invention relates to pharmaceutical compositions comprising, as active principle, at least one short-acting hypnotic agent and at least one long-acting hypnotic agent. The pharmaceutical compositions of the invention contain an effective dose of at least one short-acting hypnotic agent and of at least one long-acting hypnotic agent, or a pharmaceutically acceptable salt of these agents, a hydrate or solvate of said agents, and also at least one pharmaceutically acceptable excipient.

The excipients are chosen, according to the pharmaceutical form and the desired mode of administration, from the usual excipients known to those skilled in the art.

The short-acting hypnotic agent and the long-acting hypnotic agent may be chosen from those described hereinabove.

The pharmaceutical composition of the invention is suitable for treating and preventing sleep disorders.

In the context of the present patent application, the term “sleep disorders” especially means dyssomnia, hypersomnia, parasomnia, sleep apnea, insomnia, primary insomnia, sleep maintenance insomnia, insomnia associated with a mental disease, and insomnia induced by a drug such as caffeine, alcohol, amphetamines, opioids or anxiolytics.

The appropriate unit administration forms comprise oral-route forms such as tablets, especially coated multilayer tablets with a core, soft or hard gelatin capsules, powders, granules and oral solutions or suspensions, and sublingual or buccal administration forms.

According to one implementation form, the long-acting hypnotic agent and the short-acting hypnotic agent present in the composition according to the invention are released immediately.

According to another implementation form, the long-acting hypnotic agent present in the composition according to the invention is released immediately and the short-acting hypnotic agent is released in a sustained manner.

The immediate-release species may consist of an immediate-release unit of a pharmaceutical product, for instance an immediate-release tablet or gel capsule, or several of these units in the form of a tablet formulated in a gel capsule; the immediate-release matrix of a tablet; an immediate-release layer incorporated in a multilayer tablet; one or more coating layers in a tablet or pellet.

The sustained-release species may consist of a sustained-release unit of a pharmaceutical product, for instance a sustained-release tablet or gel capsule; or several of these units formulated in a gel capsule; a sustained-release layer incorporated in a multilayer tablet; a sustained-release core or a coating layer incorporated in a multi-coat tablet; sustained-release pellets inside a disintegrating tablet.

The long-acting hypnotic agent and the short-acting hypnotic agent may be formulated according to the invention in a single pharmaceutical composition or, alternatively, in separate pharmaceutical compositions for simultaneous, separate or sequential administration.

Via the oral route, the dose of active principle present in a composition according to the invention ranges from about 0.1 to about 30 mg of long-acting hypnotic agent and from about 0.1 to about 30 mg of short-acting hypnotic agent.

For example, a composition according to the invention contains from about 0.2 to about 15 mg and especially from 1 to 10 mg of eplivanserin in base form, and from about 0.2 to about 20 mg and especially from 1 to 10 mg of zolpidem in base form.

There may be special cases in which higher or lower dosages are suitable; such dosages are not outside the scope of the invention. According to the usual practice, the dosage that is appropriate for each patient is determined by the doctor according to the mode of administration, the weight and the response of said patient.

A first embodiment of the compositions according to the invention consists of a gel capsule comprising one or more immediate-release tablets containing the short-acting hypnotic agent and one or more immediate-release tablets containing the long-acting hypnotic agent.

Another embodiment of the compositions according to the invention consists of a gel capsule comprising one or more sustained-release tablets containing the short-acting hypnotic agent and one or more immediate-release tablets containing the long-acting hypnotic agent.

Another embodiment of the compositions according to the invention consists of a gel capsule comprising a mixture of immediate-release pellets of the short-acting hypnotic agent and of immediate-release pellets of the long-acting hypnotic agent.

Another embodiment of the compositions according to the invention consists of a gel capsule comprising a mixture of sustained-release pellets of the short-acting hypnotic agent and of immediate-release pellets of the long-acting hypnotic agent.

Another embodiment of the compositions according to the invention consists of a tablet containing immediate-release pellets of the short-acting hypnotic agent and of the long-acting hypnotic agent.

Another embodiment of the compositions according to the invention consists of a tablet containing sustained-release pellets of the short-acting hypnotic agent and immediate-release pellets of the long-acting hypnotic agent.

Another embodiment of the compositions according to the invention consists of a sustained-release enteric-coated tablet comprising immediate-release pellets of the long-acting hypnotic agent and immediate-release pellets of the short-acting hypnotic agent.

Another embodiment of the compositions according to the invention consists of a dry coated tablet comprising a sustained-release inner core containing the short-acting hypnotic agent and an immediate-release coating layer containing the long-acting hypnotic agent.

The compositions according to the invention may be prepared according to the methods known to those skilled in the art.

Thus, gel capsules comprising one or more small immediate-release tablets containing the long-acting hypnotic agent and one or more small immediate-release tablets containing the short-acting hypnotic agent may be prepared in the following manner.

The immediate-release tablets may be prepared by direct compression of mixtures of the active principles in the form of base or of salts with diluents, such as microcrystalline cellulose, mannitol, sorbitol or lactose. Other excipients, such as disintegrants or lubricants, may be added.

The choice of these functional excipients and of these diluents is well known to those skilled in the art.

According to another implementation form, the tablets may be prepared by granulation with water or with solvents of a mixture of the active principle(s) with the appropriate diluents, disintegrants and binding polymer, followed by calibration and drying of the granulate obtained and addition of a lubricant, followed by compression on a tableting machine.

The methods used are generally those described in the literature, for example B. B. Sheth, F. J. Bandelin, R. J F. Shangraw, Compressed Tablets, in Pharmaceutical Dosage Forms Tablets, Vol 1, edited by H. A. Lieberman and L Lachman, Dekker N, Y. (1980).

Gel capsules comprising one or more small immediate-release tablets containing the long-acting hypnotic agent and one or more small sustained-release tablets containing the short-acting hypnotic agent may be prepared in the following manner.

Sustained-release tablets containing the short-acting hypnotic agent may be prepared by coating immediate-release tablets as described above with a limited-diffusion polymer coating.

Polymers for this purpose may be chosen from ethylcellulose copolymers and also methyl methacrylate polymers, such as the products sold under the names Eudragit TM RS®, Eudragit TM RL® and Eudragit TM NE®.

The coating methods may consist in spraying a solution of the polymer onto the tablets, in a coating machine or a fluidized-bed device.

The solvent may be organic or aqueous, depending on the nature of the polymer used. Coating methods are described especially in J. M. Bakan, Microencapsulation, in L. Lachman, H. Lieberman and J. L. Kanig (Eds), The Theory and Practice of Industrial Pharmacy, Lea & Febinger, Philadelphia, USA, 1986; J. M. Mc Ginity, Aqueous Polymer Coatings for Pharmaceutical Dosage Forms, Dekker N.Y., 1989.

The sustained-release tablets may also be prepared by incorporating matrix-forming excipients into the formulation, without disintegrant. Examples of matrix-forming excipients are hydrophilic polymers, especially hydroxypropylmethylcellulose, hydroxymethylcellulose and hydroxyethylcellulose, which swell when they are in contact with aqueous liquids and which can control the release of the active principle through the swollen polymer network. Such excipients are used in an amount, expressed as a weight percentage, of about 10% to about 30% relative to the total weight of the tablet.

The matrix-forming excipient may also be a lipid substance, such as hydrogenated castor oil or carnauba wax, used in an amount, expressed as a weight percentage, from about 10% to about 40% relative to the total weight of the tablet.

The sustained-release tablets may be formulated, in the case of basic active principles, with a pharmaceutically acceptable organic acid chosen from those indicated below, so as to maintain the pH of the tablet during its dissolution under the neutral pH conditions of the small intestine.

Examples of organic acids that may be used include maleic acid, tartaric acid, malic acid, fumaric acid, lactic acid, citric acid, adipic acid and succinic acid, and acid salts thereof when they exist, in the form of racemates or isomers.

Gel capsules comprising a mixture of immediate-release pellets of the long-acting and short-acting hypnotic agents may be prepared in the following manner.

The immediate-release pellets of long-acting and short-acting hypnotic agents may be prepared by depositing the active principle suspended in water with, for example, hydroxypropylmethylcellulose or in an organic solvent such as ethanol, povidone or another suitable polymer acting as binder, onto a spherical granule.

A fluidized-bed coating device is generally used.

The particles may be aggregated to form spherical granules or pellets, in a high-speed granulator-mixer, or a fluidized-bed rotary agglomerator.

Such methods are described in K. W. Olson, A. M. Mehta, Int. J. Phar. Tech & Prod. Mfr. 6 18-24, 1985. The pellets may also be prepared by bulk or wet-melt extrusion followed by spheronization, as described, for example, in C. Vervaet, L. Baert & J. P. Remon, Int. J. Pharm. 116 (1995) 131-146.

The excipients used are typically those that have good plastic qualities, such as microcrystalline cellulose and mannitol. Small amounts of a polymeric binder are generally added. Surfactants such as sodium dodecyl sulfate may also be incorporated to facilitate the extrusion.

Gel capsules comprising a mixture of immediate-release pellets of the long-acting hypnotic agent and sustained-release pellets of the short-acting hypnotic agent may be prepared in the following manner.

The immediate-release pellets may be prepared as described hereinabove.

The sustained-release pellets may, in the case of basic active principles, contain a pharmaceutically acceptable organic acid or an acid salt of such an organic acid, to maintain the local pH inside the pellet throughout its dissolution under the neutral conditions of the small intestine.

Alternatively, the pellets may be coated with a pH-sensitive membrane containing a polymer that is soluble at neutral pH and impermeable at acidic pH, for instance the product Eudragit TM S®, which allows improved permeation of the active principle at pH values at and above 5, to compensate for the reduced solubility of the principle within these pH zones.

Tablets comprising several immediate-release pellets of the long-acting hypnotic agent and of the short-acting hypnotic agent may be prepared in the following manner.

The various pellets may be embedded in a matrix or the matrix itself may contain one of the hypnotic agents.

The tablets disintegrate while they are in contact with a fluid, releasing the acting principle quickly, or the immediate-release pellets, or from the coating of the immediate-release pellets.

Tablets comprising one or more immediate-release pellets of the long-acting hypnotic agent and one or more sustained-release pellets of the short-acting hypnotic agent may be prepared in the following manner.
1) The tablet may consist of a mixture of immediate-release pellets and sustained-release pellets comprising the active principles, embedded in a matrix not containing any active principle.
2) Alternatively, the pellets containing the two hypnotic agents may be embedded in a matrix which itself contains one of the two therapeutic agents.

According to another form, the sustained-release pellets may be coated with a layer comprising the active principle and excipients, allowing immediate release from this coating layer, embedded in a matrix free of active principle.

The matrix surrounding the pellets is formulated such that the compression into tablets does not interfere with the integrity of the membrane surrounding the pellets.

The tablet disintegrates while it is in contact with a fluid, releasing the long-acting agent quickly, from the matrix or from the immediate-release pellets, or from the coatings of the immediate-release pellets, and then releasing the short-acting agent, from the sustained-release pellets.

The pharmaceutical composition of the invention may also be in the form of a multilayer tablet.

Such a multilayer tablet comprises:
one or more immediate-release layers, each containing a dose of long-acting hypnotic agent and optionally a dose of short-acting hypnotic agent;
one or more sustained-release layers, each containing a dose of short-acting hypnotic agent; and
optionally an additional layer not containing any active principle but comprising hydrophilic polymers such as cellulose derivatives, for example hydroxypropylcellulose, hydroxyethylcellulose or hydroxymethylcellulose, soluble diluents such as lactose, sorbitol or mannitol, one or more other hydrophilic polymers and/or one or more other soluble excipients, this layer modifying the release of the active principle from the sustained-release layer.

Each layer optionally contains other excipients, to allow good compression, lubrication and binding of the tablet.

Another embodiment consists of a core comprising the short-acting hypnotic agent, optionally with a pharmaceutically acceptable organic acid. The core is coated with a layer of polymer containing the long-acting hypnotic agent, which is released quickly or immediately on contact with fluids, whereas the short-acting hypnotic agent is released from the core.

Optionally, the core and the coating layer may be formulated to allow release in the colon.

Each constituent of the multi-coat tablet may comprise other excipients, to allow good compression, lubrication and binding. Processes for preparing multilayer tablets and multi-coat tablets are described especially in W. C. Gunsel, Compression coated and layer tablets in pharmaceutical dosage forms: tablets, Vol 1, edited by H. A. Lieberman and L. Lachman, Dekker N.Y. (1980).

### EXAMPLE 1

### Study of the Effects of the Combination of a GABA Receptor Modulator and of a 5HT2A Receptor Inhibitor on Sleep

For this study, four groups of male Sprague-Dawley rats are used, each group comprising from 5 to 9 rats.

Group A receives eplivanserin (oral route, hemifumarate) at a dose of 3 mg/kg p.o.

Group B receives zolpidem (oral route, hemitartrate) at a dose of 3 mg/kg p.o.

Group C receives (orally) in combination 3 mg/kg p.o. of eplivanserin hemifumarate and 3 mg/kg of zolpidem hemitartrate, the two compounds being administered with an interval of 5 minutes.

Finally, group D receives zolpidem (oral route, hemitartrate) at a dose of 10 mg/kg p.o.

The data are recorded on day 0 (control day), when the animals receive only a vehicle (distilled water and methylcellulose) and on day 1 when the animals receive the active principles.

The data are recorded for 6 hours each day, the active principles being administered 15 minutes before the start of recording.

The results obtained are given in Table I below.

TABLE I

| | Group A | Group B | Group C | Group D |
|---|---|---|---|---|
| Duration of sleep | −3% | −2% | −18%** | −20%** |
| Duration of NREM | −0% | +4% | +50%** | +33%** |
| sleep | | | | |
| Lag time of | +0.6 min | −6 min | −1 min | −1.2 min |
| appearance of | | | | |
| NREM sleep | | | | |
| Mean duration of | +32%* | 15% | +58%** | +31%** |
| the periods of | | | | |
| NREM sleep | | | | |
| Mean number of | −23%* | +19%* | −5% | +1% |
| periods of NREM | | | | |
| sleep | | | | |

*p > 0.05
**p < 0.01

In Table I, the results are expressed as percentages relative to the control groups receiving only the vehicle, unless otherwise indicated.

NREM: Non-Rapid Eye Movement.

Duration of sleep: total duration of sleep during the 6 hours of recording.

Duration of NREM sleep: total duration of NREM sleep during the 6 hours of recording.

Lag time of appearance of NREM sleep: time measured from the start of the recording up to the moment of the first period of NREM sleep.

Mean duration of the periods of NREM sleep: duration of NREM sleep/number of periods of NREM sleep during the 6 hours of recording.

It is seen from Table I above that the oral dose of 3 mg/kg of eplivanserin has no effect either on the duration of sleep or on the duration of NREM sleep, but induces an increase in the mean duration of the periods of NREM sleep (and a reduction in the mean number of periods of NREM sleep).

Zolpidem at a dose of 3 mg/kg does not have a significant statistical effect on the sleep variables either, except for an increase in the mean number of periods of NREM sleep.

The combination of eplivanserin at 3 mg/kg and zolpidem at 3 mg/kg induces an increase in the NREM sleep time associated with a strong increase in the mean duration of the periods of NREM sleep, the mean number of periods of NREM sleep remaining virtually unchanged. The hypnotic effect lasts for about 3 hours in the rats.

The combination of doses of eplivanserin and zolpidem that are ineffective when they are used individually, thus makes it possible to obtain a pronounced hypnotic effect in the rats, similar to that observed with a higher dose of zolpidem used alone.

Furthermore, blockage of the 5HT2A receptors with eplivanserin promotes maintenance of the phases of NREM sleep, as is shown by the increase in the mean duration of the periods of NREM sleep.

The combination of the invention thus makes it possible to obtain a positive effect on the induction and quality of sleep, this effect not being obtained with a single hypnotic agent, even at a higher dose.

### EXAMPLE 2

### Preparation of a Gel Capsule Containing Eplivanserin and Zolpidem

A gel capsule containing, in the form of a small tablet, eplivanserin fumarate as long-acting hypnotic agent, at a dose of 1.18 mg, and zolpidem hemitartrate as short-acting hypnotic agent, at a dose of 6.22 mg, is prepared below.

The eplivanserin tablet contains the ingredients indicated in Table II below.

TABLE II

| | Percentage (%) |
|---|---|
| Ingredient | (weight/weight) |
| Micronized eplivanserin | 2.36 |
| Lactose monohydrate¹ | 87.14 |
| Gelatinized starch² | 8 |
| Sodium croscarmellose³ | 2 |
| Magnesium stearate | 0.5 |

¹Pharmatose DMV
²Starch 1500
³Ac-di-sol (FMC)

The mixture of eplivanserin fumarate, lactose monohydrate, gelatinized starch, sodium croscarmellose and sodium stearate is first prepared. The mixture is then placed in a biconical mixer for thirty minutes. The homogeneous mixture is then compressed using a standard rotary tableting machine in the form of 50-mg tablets.

The zolpidem hemitartrate tablet has the composition indicated in Table III below.

TABLE III

| | Percentage (%) |
|---|---|
| Ingredient | (weight/weight) |
| Zolpidem hemitartrate | 10.37 |
| Lactose | 83.73 |
| Microcrystalline cellulose⁴ | 10.0 |
| Hydroxypropylmethylcellulose 606⁵ | 2.1 |
| Sodium carboxymethylcellulose | 3.2 |
| Magnesium stearate | 0.6 |

⁴Avicel (FMC)
⁵Pharmacoat 606 (Shin-Etsu)

The zolpidem hemitartrate, lactose, microcrystalline cellulose, hydroxypropylmethylcellulose and sodium carboxymethylcellulose are mixed together and the mixture is then granulated with water. The granulate is then dried and calibrated. The granulate is then mixed with magnesium stearate and compressed to a mass of 60 mg per tablet, using a rotary tableting machine.

The tablets containing a 1.18-mg dose of eplivanserin fumarate and a 6.42-mg dose of zolpidem hemitartrate are then introduced into a hard gelatin gel capsule.

The dissolution profiles of the gel capsules may be measured by using a machine II from the US Pharmacopeia, with two dissolution media:
900 ml of 0.01 M hydrochloric acid, and
900 ml of 0.05 M potassium phosphate buffer at pH 6.8, maintained at 37+/−0.5° C., with stirring (50 rpm).

### EXAMPLE 3

### Preparation of a Gel Capsule Comprising an Immediate-Release Eplivanserin Tablet and a Sustained-Release Zolpidem Tablet

The immediate-release eplivanserin fumarate tablets are prepared according to the process described in Example 2 above.

The sustained-release zolpidem hemitartrate tablet is prepared according to the method described in Example 2 above to obtain a tablet having the composition indicated in Table IV below.

TABLE IV

| | Percentage (%) |
|---|---|
| Ingredients | (weight/weight) |
| Zolpidem hemitartrate | 12.4 |
| Lactose monohydrate⁶ | 33.4 |
| Hydroxypropylmethylcellulose | 25.0 |
| 4000 mPa · s⁷ | |
| Microcrystalline cellulose⁸ | 20.0 |
| Potassium hydrogen tartrate | 8.0 |
| Magnesium stearate | 1.0 |
| Colloidal anhydrous silica | 0.2 |
| Purified water | qs |

⁶Pharmatose (DMV)
⁷Metolose 90SH4000 (Shin-Etsu)
⁸Avicel PH 102 (FMC)

The same wet-granulation and compression methods as those described for zolpidem hemitartrate in Example 2 above are used. Gel capsules containing one or more 50-mg sustained-release tablets containing 5 mg of base zolpidem (corresponding to 6.22 mg of zolpidem hemitartrate) and one or more 50-mg immediate-release tablets containing 1 mg of base eplivanserin (corresponding to 1.18 mg of eplivanserin fumarate) are prepared.

The in vitro dissolution profiles of the gel capsules thus prepared may be established by using the method described in Example 2 above.

### EXAMPLE 4

### Preparation of a Gel Capsule Comprising a Mixture of Immediate-Release Eplivanserin Pellets and of Immediate-Release Zolpidem Pellets

A suspension of 59 g of eplivanserin fumarate (corresponding to 50 g of base eplivanserin) and 100 g of povidone (Pladone K29/32, BASF) in 670 g of ethanol is prepared. 750 g of this suspension are then sprayed onto 1060 g of microgranules of size 16-18 mesh, using a fluidized-bed dryer.

A suspension of 62.2 g of zolpidem tartrate (corresponding to 50 g of base zolpidem) and 100 g of povidone (Pladone K29/32, BASF) in 670 g of ethanol is then prepared. 750 g of this suspension are then sprayed onto 1060 g of microgranules of size 16-18 mesh, using an air fluidized-bed dryer.

A mixture of the two pellets is prepared, in a ratio of 1 part by weight of eplivanserin fumarate per 5 parts of zolpidem tartrate. This mixture is introduced into a hard gelatin gel capsule, to give a total amount of 1 mg of eplivanserin in base form (corresponding to 1.18 mg of eplivanserin fumarate) and 5 mg of zolpidem in base form (corresponding to 6.22 mg of zolpidem tartrate). The amount of each of the pellets may be modified to adjust the dose.

The in vitro dissolution profiles of the gel capsules thus prepared may be established using the method described in Example 2 above.

### EXAMPLE 5

### Preparation of a Gel Capsule Comprising a Mixture of Immediate-Release Eplivanserin Pellets and of Sustained-Release Zolpidem Pellets

The immediate-release eplivanserin fumarate pellets are prepared as described in Example 4 above.

Zolpidem hemitartrate pellets are prepared as described in Example 4 above.

A solution is prepared comprising 25 g of methacrylate copolymer (Eudragit TM RL 100, Rohm Pharma), 143 g of methacrylate copolymer (Eudragit TM RS 100, Rohm Pharma) and 18.7 g of ethyl citrate (Eudrafex TM, Rohm Pharma) in 1180 g of a 60/40 isopropanol/acetone mixture (weight/weight).

The zolpidem hemitartrate pellets are coated with this polymer mixture, by spraying in a fluidized-bed dryer, the final amount of coating representing 20% by weight of the mass of uncoated pellet.

After maturation of the pellets at 35° C. for 24 hours, a mixture of the coated zolpidem hemitartrate pellets and of the eplivanserin fumarate pellets in a 1:2 proportion (eplivanserin/zolpidem) is prepared, and this mixture is introduced into gelatin gel capsules to give an amount per gel capsule corresponding to 5 mg of base eplivanserin and 10 mg of base zolpidem.

The in vitro dissolution profiles of the gel capsules thus prepared may be established using the method described in Example 2 above.

### EXAMPLE 6

### Preparation of a Tablet Comprising Immediate-Release Eplivanserin Pellets and Immediate-Release Zolpidem Pellets

The eplivanserin fumarate and zolpidem hemitartrate pellets are prepared according to the method described in Example 4 above.

A mixture of the two pellets in a weight ratio of 1 part of eplivanserin fumarate per 2 parts of zolpidem hemitartrate is prepared, and 0.1% of magnesium stearate is added. The mixture is then placed in a biconical mixer for 30 minutes.

The homogeneous mixture is then tableted using a standard rotary tableting machine, to give a tablet containing 5.9 mg of eplivanserin fumarate (corresponding to 5 mg of eplivanserin in base form) and 12.44 mg of zolpidem hemitartrate (corresponding to 10 mg of zolpidem in base form).

The in vitro dissolution profiles of the gel capsules thus prepared may be established by using the method described in Example 2 above.

### EXAMPLE 7

### Preparation of a Tablet Comprising Immediate-Release Eplivanserin Pellets and Sustained-Release Zolpidem Pellets

The immediate-release eplivanserin fumarate pellets are prepared according to the process described in Example 4, and the sustained-release zolpidem pellets are prepared according to the process described in Example 5.

A mixture of the two pellets in a weight ratio of 2 parts of eplivanserin fumarate per 6 parts of zolpidem hemitartrate is prepared, and 0.2% of magnesium stearyl fumarate is added. The mixture is then transferred into a biconical mixer for 30 minutes. The homogenized mixture is then tableted using a standard rotary tableting machine, to obtain tablets containing a total amount of 4.72 mg of eplivanserin fumarate (corresponding to 4 mg of base eplivanserin) and 14.93 mg of zolpidem hemitartrate (corresponding to 12 mg of base zolpidem).

The in vitro dissolution profiles of the gel capsules thus prepared may be established using the method described in Example 2 above.

### EXAMPLE 8

### Preparation of a Sustained-Release Enteric-Coated Tablet Comprising Immediate-Release Eplivanserin Pellets and Immediate-Release Zolpidem Pellets

Tablets comprising both eplivanserin fumarate and zolpidem hemitartrate are prepared according to the process described in Example 6.

The tablets are then coated according to the process described below.

A solution of 46 g of methacrylate copolymer (Eudragit TM RL100, Rohm Pharma), 295 g of methacrylate copolymer (Eudragit TM RS100, Rohm Pharma) and 40 g of ethyl citrate (Eudrafex TM, Rohm Pharma) in 2280 g of a 65/35 isopropanol/acetone mixture (weight/weight) is prepared.

The tablets comprising 3.93 mg of eplivanserin fumarate and 12.44 mg of zolpidem hemitartrate are coated with the polymer mixture, by spraying in a system of “coating pan” type, the final amount of coating being from 5% to 10% by weight of the mass of pellet without coating.

### EXAMPLE 9

### Preparation of a Two-Layer Tablet Comprising an Immediate-Release Eplivanserin Layer and an Immediate-Release Zolpidem Layer

Granulates A are prepared by dry-mixing and granulates B are prepared by wet-mixing as described in Example 2, and according to the compositions indicated in Table V below.

TABLE V

| | Percentage (%) |
|---|---|
| Ingredients | (weight/weight) |
| Granulates A | |
| Eplivanserin fumarate | 2.95 |
| Dry lactose monohydrate⁹ | 82.71 |
| Pregelatinized starch¹⁰ | 8.00 |
| Croscarmellose¹¹ | 2.00 |
| Sodium carboxymethylcellulose¹² | 3.80 |
| Magnesium stearate¹³ | 0.54 |
| Granulates B | |
| Zolpidem hemitartrate | 6.22 |
| Lactose monohydrate⁹ | 73.88 |
| Microcrystalline cellulose¹⁴ | 14.0 |
| Hydroxypropylmethylcellulose 606¹⁵ | 2.1 |
| Sodium carboxymethylcellulose¹² | 3.2 |
| Magnesium stearate¹³ | 0.6 |

⁹Pharmatose (DMV)
¹⁰Starch 1500 (Colorcon)
¹¹Ac-di-sol (FMC)
¹²Blanose (Aqualon)
¹³Brentag AG
¹⁴Avicel PH 102 (FMC)
¹⁵Pharmacoat 606 (Shin-Etsu)

The mixtures are then tableted as a two-layer tablet using an alternating tableting machine, the first immediate-release layer of a mass of 200 mg of granulate A comprising 5.90 mg of eplivanserin fumarate (corresponding to 5 mg of base eplivanserin) and the second immediate-release layer of a mass of 200 mg of granulate B comprising 12.44 mg of zolpidem hemitartrate (corresponding to 10 mg of base zolpidem).

The in vitro dissolution profiles of the gel capsules thus prepared may be established by using the method described in Example 2 above.

### EXAMPLE 10

### Preparation of a Two-Layer Tablet Comprising an Immediate-Release Eplivanserin Layer and a Sustained-Release Zolpidem Layer

Granulates C are prepared by dry-mixing and granulates D are prepared by wet-mixing as described in Example 2 and according to the compositions indicated in Table VI below.

TABLE VI

| | Percentage (%) |
|---|---|
| Ingredients | (weight/weight) |
| Granulates C | |
| Eplivanserin fumarate | 2.95 |
| Dry lactose monohydrate¹⁶ | 84.00 |
| Pregelatinized starch¹⁷ | 7.70 |
| Croscarmellose¹⁸ | 2.00 |
| Sodium carboxymethylcellulose¹⁹ | 3.4 |
| Magnesium stearate²⁰ | 0.54 |
| Granulates D | |
| Zolpidem hemitartrate | 7.75 |
| Lactose 150 mesh¹⁶ | 37.85 |
| Microcrystalline cellulose²¹ | 20.0 |
| Tartaric acid (23) | 8.4 |
| Hydroxypropylmethylcellulose²² | 25.0 |
| Magnesium stearate²³ | 1.0 |

¹⁶Pharmatose (DMV)
¹⁷Starch 1500 (Colorcon)
¹⁸Ac-di-sol (FMC)
¹⁹Blanose (Aqualon)
²⁰Brentag AG
²¹Avicel PH 102 (FMC)
²²Metolose 90SH4000 (Shin-Etsu)
²³Brentag AG

The mixtures are tableted as a two-layer tablet using an alternating tableting machine, the first immediate-release layer of a mass of 150 mg of granulate C comprising 4.425 mg of eplivanserin fumarate (corresponding to 3.75 mg of base eplivanserin) and the second sustained-release layer of a mass of 200 mg of granulate D comprising 15.50 mg of zolpidem hemitartrate (corresponding to 12.45 mg of base zolpidem).

The in vitro dissolution profiles of the gel capsules thus prepared may be established using the method described in Example 2 above.

### EXAMPLE 11

### Preparation of a Three-Layer Tablet Comprising an Immediate-Release Eplivanserin Layer, an Inactive Layer and a Sustained-Release Zolpidem Third Layer

Granulates E and F are prepared by dry-mixing and granulates G are prepared by wet-mixing as described in Example 2 and according to the compositions indicated in Table VII below.

TABLE VII

| | Percentage (%) |
|---|---|
| Ingredients | (weight/weight) |
| Granulates E (immediate release) | |
| Eplivanserin fumarate | 2.36 |
| Dry lactose monohydrate²⁴ | 87.14 |
| Pregelatinized starch²⁵ | 8.0 |
| Croscarmellose²⁶ | 2.0 |
| Sodium carboxymethylcellulose²⁷ | 3.8 |
| Magnesium stearate²⁸ | 0.54 |
| Granulates F (inactive) | |
| Dry lactose monohydrate²⁴ | 60.0 |
| Microcrystalline cellulose²⁹ | 24.0 |
| Tartaric acid³⁰ | 10.0 |
| Hydroxyethylcellulose | 5.0 |
| Magnesium stearate²⁸ | 1.0 |
| Granulates G (sustained release) | |
| Zolpidem hemitartrate | 5.0 |
| Lactose 200 mesh²⁴ | 67.7 |
| Microcrystalline cellulose²⁹ | 20.0 |
| Hydroxypropylmethylcellulose 606³¹ | 2.5 |
| Sodium carboxymethylcellulose²⁷ | 3.8 |
| Magnesium stearate²⁸ | 1.0 |

²⁴Pharmatose (DMV)
²⁵Starch 1500 (Colorcon)
²⁶Ac-di-sol (FMC)
²⁷Blanose (Aqualon)
²⁸Brentag AG
²⁹Avicel PH 102 (FMC)
³⁰Brentag AG
³¹Pharmacoat (Shin-Etsu)

The mixtures as described in Example 9 are tableted as a three-layer tablet, an outer layer with a mass of 125 mg of granulate E comprising 2.95 mg of eplivanserin fumarate (corresponding to 2.5 mg of base eplivanserin), an intermediate layer with a mass of 125 mg of granulate F and a third outer layer with a mass of 300 mg of granulate G comprising 15 mg of zolpidem hemitartrate (corresponding to 12.06 mg of base zolpidem).

### EXAMPLE 12

### Preparation of a Dry Coated Tablet Comprising a Zolpidem Inner Core and an Eplivanserin Outer Coating

Granulates are prepared in the manner described in Example 2, on the basis of the compositions indicated in Table VIII below.

TABLE VIII

| | Percentage (%) |
|---|---|
| Ingredients | (weight/weight) |
| Inner core (sustained release) | |
| Zolpidem hemitartrate | 15.55 |
| Lactose monohydrate 200 mesh³² | 36.05 |
| Microcrystalline cellulose³³ | 18.0 |
| Hydroxypropylmethylcellulose³⁴ | 21.0 |
| Tartaric acid³⁵ | 8.4 |
| Magnesium stearate³⁵ | 1.0 |
| Outer coating (immediate release) | |
| Eplivanserin fumarate | 1.96 |
| Lactose monohydrate 150 mesh³² | 52.00 |
| Microcrystalline cellulose³³ | 39.84 |
| Hydroxypropylmethylcellulose 606³⁴ | 2.2 |
| Sodium carboxymethylcellulose³⁶ | 3.0 |
| Magnesium stearate³⁵ | 1.0 |

³²Pharmatose (DMV)
³³Avicel PH 102 (FMC)
³⁴Metolose 90SH4000 (Shin-Etsu)
³⁵Brentag AG
³⁶Blanose (Aqualon)

The granulate forming the inner core is tableted as small tablets using an alternating tableting machine, before performing the dry-coating operation with the second layer. This operation gives 80-mg sustained-release tablets containing 12.44 mg of zolpidem hemitartrate (corresponding to 10 mg of base zolpidem).

The granulate forming the outer coating layer is tableted using a rotary tableting machine, making it possible to include the small tablets of inner core. The outer layer has a mass of 301 mg and contains 5.9 mg of eplivanserin fumarate (corresponding to 5 mg of base eplivanserin).

According to another of its aspects, a subject of the invention is the use of at least one long-acting hypnotic agent in combination with at least one short-acting hypnotic agent, for the preparation of a medicament for preventing and/or treating sleep disorders as described hereinabove, especially insomnia.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A combination of at least one short-acting hypnotic agent chosen from zolpidem, zopiclone, eszopiclone, zaleplon, melatonin, ramelteon, triazolam, etizolam, brotizolam and indiplon, or a salt, hydrate or solvate, or a mixture in any combination thereof with at least one long-acting hypnotic agent chosen from eplivanserin, temazepam, clonazepam, gaboxadol and pregabaline, or an addition salt, hydrate or solvate, or a mixture in any combination thereof.

2. The combination as claimed in claim 1, wherein the short-acting hypnotic agent is present in a galenical formulation suitable for immediate or sustained release, and the long-acting hypnotic agent is present in a galenical formulation suitable for immediate release.

3. A pharmaceutical composition comprising, as active principle, at least one short-acting hypnotic agent chosen from zolpidem, zopiclone, eszopiclone, zaleplon, melatonin, ramelteon, triazolam, etizolam, brotizolam and indiplon, or a pharmaceutically acceptable salt, hydrate or solvate, or a mixture in any combination thereof, and at least one long-acting hypnotic agent chosen from eplivanserin, temazepam, clonazepam, gaboxadol and pregabaline, or a pharmaceutically acceptable salt, hydrate or solvate, or a mixture in any combination thereof, in combination with at least one pharmaceutically acceptable excipient.

4. The composition as claimed in claim 3, wherein the short-acting hypnotic agent is zolpidem or a salt thereof and the long-acting hypnotic agent is eplivanserin or a salt thereof.

5. The composition as claimed in claim 3, wherein the short-acting hypnotic agent and the long-acting hypnotic agent are released immediately.

6. The composition as claimed in claim 4, wherein the short-acting hypnotic agent and the long-acting hypnotic agent are released immediately.

7. The composition as claimed in claim 3, wherein the short-acting hypnotic agent is released in a sustained manner and in that the long-acting hypnotic agent is released immediately.

8. The composition as claimed in claim 4, wherein the short-acting hypnotic agent is released in a sustained manner and in that the long-acting hypnotic agent is released immediately.

9. The composition as claimed in claim 3, wherein it consists of a gel capsule comprising one or more immediate-release tablets containing the short-acting hypnotic agent and one or more immediate-release tablets containing the long-acting hypnotic agent.

10. The composition as claimed in claim 4, wherein it consists of a gel capsule comprising one or more immediate-release tablets containing the short-acting hypnotic agent and one or more immediate-release tablets containing the long-acting hypnotic agent.

11. The composition as claimed in claim 3, wherein it consists of a gel capsule containing one or more sustained-release tablets containing the short-acting hypnotic agent and one or more immediate-release tablets containing the long-acting hypnotic agent.

12. The composition as claimed in claim 4, wherein it consists of a gel capsule containing one or more sustained-release tablets containing the short-acting hypnotic agent and one or more immediate-release tablets containing the long-acting hypnotic agent.

13. The composition as claimed in claim 3, wherein it consists of a gel capsule containing a mixture of immediate-release pellets of the short-acting hypnotic agent and of immediate-release pellets of the long-acting hypnotic agent.

14. The composition as claimed in claim 4, wherein it consists of a gel capsule containing a mixture of immediate-release pellets of the short-acting hypnotic agent and of immediate-release pellets of the long-acting hypnotic agent.

15. The composition as claimed in claim 3, wherein it consists of a gel capsule comprising a mixture of sustained-release pellets of the short-acting hypnotic agent and of immediate-release pellets of the long-acting hypnotic agent.

16. The composition as claimed in claim 4, wherein it consists of a gel capsule comprising a mixture of sustained-release pellets of the short-acting hypnotic agent and of immediate-release pellets of the long-acting hypnotic agent.

17. The composition as claimed in claim 3, wherein it consists of a tablet containing immediate-release pellets of the short-acting hypnotic agent and of the long-acting hypnotic agent.

18. The composition as claimed in claim 4, wherein it consists of a tablet containing immediate-release pellets of the short-acting hypnotic agent and of the long-acting hypnotic agent.

19. The composition as claimed in claim 3, wherein it consists of a tablet containing sustained-release pellets of the short-acting hypnotic agent and immediate-release pellets of the long-acting hypnotic agent.

20. The composition as claimed in claim 4, wherein it consists of a tablet containing sustained-release pellets of the short-acting hypnotic agent and immediate-release pellets of the long-acting hypnotic agent.

21. The composition as claimed in claim 3, wherein it consists of a sustained-release enteric-coated tablet comprising immediate-release pellets of the long-acting hypnotic agent and immediate-release pellets of the short-acting hypnotic agent.

22. The composition as claimed in claim 4, wherein it consists of a sustained-release enteric-coated tablet comprising immediate-release pellets of the long-acting hypnotic agent and immediate-release pellets of the short-acting hypnotic agent.

23. The composition as claimed in claim 3, wherein it consists of a multilayer tablet comprising:
(a) one or more immediate-release layers, each containing a dose of long-acting hypnotic agent and optionally a dose of short-acting hypnotic agent,
(b) one or more sustained-release layers, each containing a dose of short-acting hypnotic agent, and optionally
(c) an inactive layer not containing any hypnotic agent.

24. The composition as claimed in claim 4, wherein it consists of a multilayer tablet comprising:
(a) one or more immediate-release layers, each containing a dose of long-acting hypnotic agent and optionally a dose of short-acting hypnotic agent,
(b) one or more sustained-release layers, each containing a dose of short-acting hypnotic agent, and optionally
(c) an inactive layer not containing any hypnotic agent.

25. The composition as claimed in claim 3, wherein it consists of a dry-coated tablet, which comprises an inner sustained-release core containing the short-acting hypnotic agent and in that the immediate-release coating layer contains the long-acting hypnotic agent.

26. The composition as claimed in claim 4, wherein it consists of a dry-coated tablet, which comprises an inner sustained-release core containing the short-acting hypnotic agent and in that the immediate-release coating layer contains the long-acting hypnotic agent.

27. A method of treating a sleep disorder in a patient comprising administering to said patient a therapeutically effective amount of a combination consisting of at least one short-acting hypnotic agent chosen from zolpidem, zopiclone, eszopiclone, zaleplon, melatonin, ramelteon, triazolam, etizolam, brotizolam and indiplon, or a pharmaceutically acceptable salt, or a hydrate or a solvate thereof, and at least one long-acting hypnotic agent chosen from eplivanserin, temazepam, clonazepam, gaboxadol and pregabaline, or a pharmaceutically acceptable salt, a hydrate or a solvate thereof.

28. The method according to claim 27, wherein the sleep disorder is dyssomnia, hypersomnia, parasomnia, sleep apnea, insomnia, primary insomnia, sleep maintenance insomnia, insomnia associated with a mental disease, and insomnia induced by caffeine, alcohol, amphetamines, opioids or anxiolytics.

29. The method according to claim 27, wherein the sleep disorder is sleep maintenance insomnia.

30. The method according to claim 27, wherein the short acting hypnotic is zolpidem or a salt thereof and the long acting hypnotic is eplivanserin or a salt thereof.
